(19) United States
(12) Patent Application Publication
(10) Pub. No.: US 2017/0189474 A1
(43) Pub. Date: Jul. 6, 2017

(54) ANTIFUNGAL THERAPY FOR THE TREATMENT OF HIRSCHSPRUNG-ASSOCIATED ENTEROCOLITIS

(71) Applicant: Cedars-Sinai Medical Center, Los Angeles, CA (US)

(72) Inventor: Philip K. FRYKMAN, Los Angeles, CA (US)

(73) Assignee: Cedars-Sinai Medical Center, Los Angeles, CA (US)

(21) Appl. No.: 15/461,349

(22) Filed: Mar. 16, 2017

Related U.S. Application Data
(63) Continuation-in-part of application No. PCT/US2015/050662, filed on Sep. 17, 2015.
(60) Provisional application No. 62/052,357, filed on Sep. 18, 2014.

(51) Int. Cl. A61K 38/14; A61K 31/496; A61K 31/4164; A61K 31/4196; A61K 31/4439; C12Q 1/68; A61K 31/7048

(52) U.S. Cl. CPC A61K 38/14; C12Q 1/6895; A61K 31/496; A61K 31/7048; A61K 31/4196; A61K 31/4439; A61K 31/4164; C12Q 2600/106; C12Q 2600/158

(57) ABSTRACT

The present invention provides for methods and systems for the treatment and diagnosis of Hirschsprung-associated enterocolitis. Treatment of Hirschsprung-associated enterocolitis can include providing and administering an antifungal agent or a combination of an antifungal agent and an antibiotic. Diagnosis of Hirschsprung-associated enterocolitis can include the detection of fungal species by way of qPCR.

### CROSS-REFERENCE TO RELATED APPLICATIONS

This application is a continuation-in-part of International Application No. PCT/US2015/050662, filed Sep. 17, 2015, which designated the U.S. and that International Application was published under PCT Article 21(2) in English, which claims priority under 35 U.S.C. §119(e) to U.S. provisional patent application No. 62/052,357, filed Sep. 18, 2014.

### STATEMENT REGARDING FEDERALLY-SPONSORED RESEARCH OR DEVELOPMENT

This invention was made with government support under Grant Nos. TR000124, DK090281, and DK104040 awarded by National Institutes of Health. The government has certain rights in the invention.

### FIELD OF INVENTION

This invention relates to the diagnosis and treatment of Hirschsprung-associated enterocolitis.

### BACKGROUND

The following description includes information that may be useful in understanding the present invention. It is not an admission that any of the information provided herein is prior art or relevant to the presently claimed invention, or that any publication specifically or implicitly referenced is prior art.

Congenital aganglionic megacolon, more commonly known as Hirschsprung disease (HSCR), was first described by Harald Hirschsprung in 1887. The hallmark pathological feature of this condition is the absence of ganglion cells in the distal colon causing a functional bowel obstruction in newborns. Nowadays most infants with HSCR are treated with colon pull-through surgery that removes the aganglionic portion of colon and re-establishes fecal continuity in the first weeks of life. Surgical results are satisfactory for most children, however 20-30% of children experience a serious and potentially life-threatening enterocolitis after surgery. Even today, Hirschsprung-associated enterocolitis (HAEC) remains the most frequent complication in children with HSCR resulting in frequent hospitalizations and is the primary cause of mortality in this population.

While many etiologies have been proposed for HAEC, the underlying biological mechanisms are poorly understood. A microbial role in the development of HAEC has been suspected since it was first described over 50 years, although at present, no specific organisms have been identified. Both Clostridium difficile and rotavirus have been implicated as causative agents of HAEC, however neither were consistently present in patients HAEC. With the advancement of molecular microbiological techniques, a PCR based methodology demonstrated that colonization of Bifidobacteria and Lactobacilli genera were decreased in HSCR patients who developed HAEC, compared with those who did not develop HAEC, suggesting that the composition of the bacterial populations may play a role HAEC. Using a genomics approach, Yan et al. recently reported characterization of the colonic bacterial microbiome of 4 infants with HSCR, two of which had HAEC. Fecal specimens from multiple regions of the colon were obtained at the time of surgery and showed increased bacterial population diversity in the HAEC patients compared with HSCR patients. These early data suggest the possibility that HSCR children who develop HAEC have a dysbiosis.

Still, little is known about the underlying mechanisms leading to this enterocolitis. Accordingly, there remains a need in the art for methods and systems of diagnosis and treatment of HAEC.

### SUMMARY OF THE INVENTION

The following embodiments and aspects thereof are described and illustrated in conjunction with compositions and methods which are meant to be exemplary and illustrative, not limiting in scope.

Various embodiments of the present invention provide for a method of treating Hirschsprung-associated enterocolitis, comprising providing an antifungal agent; and administering the antifungal agent to a subject in need thereof.

In various embodiments, the antifungal agent can be isavuconazonium sulfate, posaconazole, itraconazole, efinaconazole, tavaborole, luliconazole, terbinafine, auriclosene, E-1224, VT-1161, NDV-3, NDV-3A, SQ-109, MGCD-290, ME-1111, LACTIN-V, or combinations thereof, or salts thereof. In various embodiments, the antifungal agent can be natamycin, fluconazole or a combination thereof, or salts thereof.

In various embodiments, the Hirschsprung-associated enterocolitis can be caused, at least in part, by a fungus. In various embodiments, the Hirschsprung-associated enterocolitis can be caused, at least in part, by Candida.

In various embodiments, the method can further comprise providing an antibiotic agent and administering the antibiotic agent.

In various embodiments, the antibiotic agent can be selected from the group consisting of metronidazole, vancomycin, gentamicin, ciprofloxacin, levofloxacin and combinations thereof. In various embodiments, the antibiotic agent is a combination of piperacillin and tazobactam.

Various embodiments of the present invention provide for a method of diagnosing Hirschsprung-associated enterocolitis, comprising providing a biological sample; quantitating the amount of a fungal species in the biological sample; diagnosing Hirschsprung-associated enterocolitis when the quantity of the fungal species is higher than a reference value.

Various embodiments of the present invention provide for a method of selecting a treatment for a subject suspected of having Hirschsprung-associated enterocolitis, comprising providing a biological sample; quantitating the amount of a fungal species in the biological sample; selecting an antifungal treatment for the Hirschsprung-associated enterocolitis when the quantity of the fungal species is higher than a reference value.

In various embodiments, quantitating the amount of a fungal species in the biological sample can comprise using quantitative polymerase chain reaction (qPCR) detection of the amount of the fungal species.

In various embodiments, the fungal species can be a Candida species. In various embodiments, the fungal species can be a Saccharomyces species.

In various embodiments, quantitating the amount of a fungal species in the biological sample can comprise using quantitative polymerase chain reaction (qPCR) detection of the amount of the fungal species and TTTATCAACTTGTCACACCAGA (SEQ ID NO:1) (Forward) and ATCCCGCCTTACCACTACCG (SEQ ID NO:2) (Reverse) are used for C. albicans, and CAATCCTACCGCCAGAGGTTAT (SEQ ID NO:3) (Forward) and TGGCCACTAGCAAAATAAGCG (SEQ ID NO:4) (Reverse) can be used for C. tropicalis.

In various embodiments, quantitating the amount of a fungal species in the biological sample can comprise using quantitative polymerase chain reaction (qPCR) detection of the amount of the fungal species and one or more primers selected from the group consisting of SEQ ID NO:1, SEQ ID NO:2, SEQ ID NO:3, SEQ ID NO:4, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20.

In various embodiments, quantitating the amount of a fungal species in the biological sample can comprise using next generation sequencing (NGS).

In various embodiments, the antifungal treatment can comprise an antifungal agent. In various embodiments, the antifungal agent can be selected from the group consisting of natamycin, fluconazole, isavuconazonium sulfate, posaconazole, itraconazole, efinaconazole, tavaborole, luliconazole, terbinafine, auriclosene, E-1224, VT-1161, NDV-3, NDV-3A, SQ-109, MGCD-290, ME-1111, LACTIN-V, combinations thereof, and salts thereof.

In various embodiments, the methods can further comprising selecting an antibiotic.

In various embodiments, the antibiotic can be selected from the group consisting of metronidazole, vancomycin, gentamicin, ciprofloxacin, levofloxacin and combinations thereof. In various embodiments, the antibiotic can be a combination of piperacillin and tazobactam.

Other features and advantages of the invention will become apparent from the following detailed description, taken in conjunction with the accompanying drawings, which illustrate, by way of example, various features of embodiments of the invention.

### BRIEF DESCRIPTION OF THE FIGURES

Exemplary embodiments are illustrated in referenced figures. It is intended that the embodiments and figures disclosed herein are to be considered illustrative rather than restrictive.

FIGS. 1A-1B depict bacterial phyla in HSCR and HAEC patients in accordance with various embodiments of the present invention. 1A,16S rRNA gene sequence of fecal bacteria of nine HSCR patients and nine HAEC patients. The pie charts show average relative abundance of five major phyla and six subdominant phyla (summarized as “All others”). 1B, histograms demonstrating the phyla level bacterial composition of individual subjects with HSCR and HAEC. Individual subject numbers are labeled on the X axis and expressed as relative OTU abundance per each subject.

FIGS. 2A-2B show that fungal genera of HAEC patients have increased Candida sp. abundance compared with HSCR patients in accordance with various embodiments of the present invention. 2A, Genera level distribution of fungi in nine HSCR patients and eight HAEC patients expressed as OTU abundance of 18S ITS-1 sequences (upper panel). Candida species composition in HSCR and HAEC patients (lower panel). 2B, Histograms demonstrating the fungal genera composition of individual subjects with HSCR and HAEC. Individual subject numbers are labeled on the X axis and expressed as relative OTUs abundance per each subject. Seventy-four different genera were identified by ITS-1 sequencing. The histogram shows 13 most abundant genera, unclassified genera, and 61 infrequent genera being summarized as “All others”.

FIG. 3 depicts quantitative PCR of Candida albicans in accordance with various embodiments of the present invention. The quantitation of C. albicans by quantitative PCR on total fecal DNA from HSCR and HAEC patients.

FIGS. 4A-4B depict Candida albicans and tropicalis OTU abundance by phenotype in accordance with various embodiments of the present invention. 4A, The OTU abundance of Candida in feces of HSCR and HAEC patients. Three out of eight HAEC patients showed very elevated Candida OTU's. 4B, relative distribution of C. albicans to C. tropicalis in the “high burden” patients compared with the “low burden” patients.

FIG. 5 depicts rarefaction curves of sequencing data in accordance with various embodiments of the present invention. Rarefaction curves showing the Shannon diversity index change with increasing sequencing depth show that the bacterial (top) and fungal (bottom) sequencing of samples from HSCR patients (left) and HAEC patients (right) reached saturated plateau phase. The plateau in each curve estimates the minimum number of sequences necessary to capture diversity.

FIG. 6 depicts correlation analysis between qPCR method in accordance with embodiments of the present invention and culture-based method, indicating that correlation coefficients r=0.935. Scatter with smooth lines and markers to compare pairs of values.

FIG. 7 depicts correlation analysis between qPCR method in accordance with embodiments of the present invention and culture-based method, wherein patient 03-0002 was excluded, which indicate correlation coefficients r=1.0. Scatter with smooth lines and markers to compare pairs of values.

FIG. 8 depicts an exemplary device/system in accordance with various embodiments of the present invention.

FIG. 9 depicts changes in enterocolitis severity following medicated water treatments, in accordance with embodiments of the present invention. 129SvEdnrb−/− (KO) mice were treated with antibiotics (KO_ABX, n=5) or antifungal agents (KO_AFX1 and KO_AFX2, n=6 for each) alone, and combination with both (KO_ABX+AFX1, KO_ABX+AFX2; n=6 and 7, respectively). Regular water treated KO mice (KO_H₂O) and their wild type littermates (WT_H₂O) were used as untreated control. The KO_H₂O mice had a significant higher enterocolitis score (4.286±0.9184) than WT_H₂O without any enterocolitis. The total enterocolitis score of each treatment was compared with KO_H₂O by using unpaired two-tailed Student's t-test. Results are shown as Mean±SEM, *P<0.05; **P<0.01.

FIG. 10 depicts fecal fungal communities for each treatment group and control, in accordance with embodiments of the present invention. ITS region gene sequence of fecal fungi of 129SvEdnrb+/+ (WT) and −/− (KO) with different medicated water treatments. The pie charts show average relative abundance of five major phyla.

FIG. 11 depicts Ednrb mice, microsurgical pull-through operation and post-surgical enterocolitis. Panel 1, Ednrb+/+ and Ednrb−/− mice with enterectomy showing megacolon (a) and aganglionic region (b). Panel 2 shows murine colon pull-through operation A, resection of aganglionic region and megacolon; B, preparation of pull-through segment; C, completed pull-through operation. Panel 3 Enterocolitis scores of healthy and clinically ill post-pullthrough Ednrb−/− mice.

### DESCRIPTION OF THE INVENTION

Unless defined otherwise, technical and scientific terms used herein have the same meaning as commonly understood by one of ordinary skill in the art to which this invention belongs. Singleton et al., Dictionary of Microbiology and Molecular Biology 3rd ed., Revised, J. Wiley & Sons (New York, N.Y. 2006); March, Advanced Organic Chemistry Reactions, Mechanisms and Structure 7^{th} ed., J. Wiley & Sons (New York, N.Y. 2013); and Sambrook and Russel, Molecular Cloning: A Laboratory Manual 4^{th} ed., Cold Spring Harbor Laboratory Press (Cold Spring Harbor, N.Y. 2012), provide one skilled in the art with a general guide to many of the terms used in the present application.

One skilled in the art will recognize many methods and materials similar or equivalent to those described herein, which could be used in the practice of the present invention. Indeed, the present invention is in no way limited to the methods and materials described. For purposes of the present invention, the following terms are defined below.

“Mammal” as used herein refers to any member of the class Mammalia, including, without limitation, humans and nonhuman primates such as chimpanzees, and other apes and monkey species; farm animals such as cattle, sheep, pigs, goats and horses; domestic mammals such as dogs and cats; laboratory animals including rodents such as mice, rats and guinea pigs, and the like. The term does not denote a particular age or sex. Thus adult, pediatric and newborn subjects, as well as fetuses, whether male or female, are intended to be including within the scope of this term.

“Selecting a therapy” as used herein refers to, for example, recommending, picking, choosing, directing or prescribing a therapy to the subject.

Candida albicans is an opportunistic fungal pathogen found as part of the normal microflora in the human digestive tract that can cause a wide spectrum of disease. Our understanding of the role of commensal fungi in human health and disease is in its infancy at present, however with advanced genomics and bioinformatic methodology, it is advancing rapidly. An example of dramatic differences in the fungal microbiome, referred to as the “mycobiome”, in health and disease was illuminated in our study of children with Hirschsprung disease who had a history of enterocolitis compared with those who had never had enterocolitis. These differences implicate a role for C. albicans as a pathogen in patients with enterocolitis and a novel therapeutic target. To determine which patients may benefit from a novel therapy (antifungals) for Hirschsprung-associated enterocolitis, we developed a rapid diagnostic test to detect and quantify specific fungal species in human fecal specimens using quantitative PCR.

A rapid qPCR detection of fungal species C. albicans, C. tropicalis, and Saccharomyces cerevisiae was developed. The PCR amplification of the Candida was performed by using specific primers flanked Candida albicans internal transcribed spacer (ITS) region. The QIAamp DNA stool kit provides a fast pretreatment procedure for extracting DNA from fecal samples in order to introduce specific, and more sensitive tool than fecal culture and to improve diagnosis and management of invasive candidiasis.

Herein, we studied 18 HSCR patients from four centers, all of whom had completed definitive surgery to treat HSCR. We compared the colonic bacterial and fungal populations in 9 children with HSCR alone, with 9 children who had at least one episode of HAEC.

The most striking finding in the study described herein is the high burden of C. albicans in some HAEC patients, which was not found in the HSCR group. Heretofore, fecal fungi have not been studied in Hirschsprung patients, nor has fungi been implicated in playing a role in HAEC. Importantly, we could not identify a shared clinical feature among high fungal burden HAEC patients, in terms of age, location of transition zone, diet, probiotic use, complications or trisomy 21 status. Prior treatment with systemic antibiotics is a potential explanation, as antibiotics are known to result in intestinal Candida blooms. However, recent antibiotic administration does not fully explain these findings because while 2 high burden patients had received antibiotics, so did 2 low burden patients as well (Table 2).

Further, this is the largest study to date comparing the bacterial microbiome composition of children with HSCR to those who had a history of HAEC that demonstrated modest but potentially important differences. The HAEC group showed reduced Firmicutes of which the largest represented genera is the probiotic microbe Lactobacillus, which is similar to the findings of Shen et al. demonstrating reduced Lactobacillus in HSCR and HAEC patients compared with control. In addition, the HAEC patients showed a marked reduction in Verrucomicrobia, of which Akkermansia muciniphilia is the single most frequently, represented species. A. muciniphilia has been proposed to have a protective or anti-inflammatory role in the mucous barrier, and our findings of reduced Verrucomicrobia in the HAEC patients are similar to those of patients with acute appendicitis and inflammatory bowel disease (IBD) and no statistically significant differences between groups were noted in the bacterial genera. These findings suggest similarities in the gut bacterial milieu of HAEC to IBD, which is intriguing given the recent reports of Hirschsprung disease patients with suspected chronic HAEC have been diagnosed with IBD. These findings suggest a dysequilibrium in the gut microbial ecosystem of HAEC patients, such that there may be dominance of bacteria and fungi predisposing patients to development of HAEC.

While not wishing to be bound by any particular theory, we believe that there may be a subset of children with HAEC in whom C. albicans may be either a commensal species that is expanded as a consequence of enterocolitis (or treatment), or the that C. albicans is a pathobioant that contribute to the pathogenesis of HAEC. While the mechanism leading to the C. albicans expansion is unclear, it can be an underlying defect in the gut innate immunity of Hirschsprung patients that may predispose these patients to developing HAEC. Based on these findings, antifungal therapy is provided herein as a therapy in selected patients with HAEC.

### Treatments

Various embodiments of the present invention provide for a method of treating Hirschsprung-associated enterocolitis.

In various embodiments, the method comprises providing an antifungal agent; and administering the antifungal agent to a subject in need thereof to treat Hirschsprung-associated enterocolitis.

In various embodiments, the method comprises providing an antifungal agent and an antibiotic; and administering the antifungal agent and the antibiotic to a subject in need thereof to treat Hirschsprung-associated enterocolitis.

Various embodiments of the present invention provide of a method of inhibiting or reducing the growth of a fungal species in a subject having or suspected of having Hirschsprung-associated enterocolitis.

In various embodiments, the method comprises providing an antifungal agent; and administering the antifungal agent to the subject who has or is suspected to have Hirschsprung-associated enterocolitis to inhibit or reduce the growth of the fungal species.

In various embodiments, the method comprises providing an antifungal agent and an antibiotic; and administering the antifungal agent and the antibiotic to the subject who has or is suspected of having Hirschsprung-associated enterocolitis to inhibit or reduce the growth of the fungal species.

Various embodiments of the present invention provide for a method of reducing the likelihood of Hirschsprung-associated enterocolitis.

In various embodiments, the method comprises providing an antifungal agent; and administering the antifungal agent to a subject in need thereof to reduce the likelihood of having Hirschsprung-associated enterocolitis.

In various embodiments, the method comprises providing an antifungal agent and an antibiotic; and administering the antifungal agent and the antibiotic to a subject in need thereof to reduce the likelihood of having Hirschsprung-associated enterocolitis.

Various embodiments of the present invention provide for a method of inhibiting or reducing the growth a fungal species in a subject who is susceptible of developing Hirschsprung-associated enterocolitis.

In various embodiments, the method comprises providing an antifungal agent; and administering the antifungal agent to the subject who is susceptible of developing Hirschsprung-associated enterocolitis to inhibit or reduce the growth the fungal species.

In various embodiments, the method comprises providing an antifungal agent and an antibiotic; and administering the antifungal agent and the antibiotic to to the subject who is susceptible of developing Hirschsprung-associated enterocolitis to inhibit or reduce the growth the fungal species.

In various embodiments, the Hirschsprung-associated enterocolitis is caused, at least in part, by C. albicans, C. tropicalis, or Saccharomyces cerevisiae.

### Subjects

In various embodiments, the subject who is treated for HAEC or for whom the likelihood of HAEC is reduced is a child. In various embodiments, the child is under 18 years of age. In various embodiments, the child is 13 years of age or less. In various embodiments, the subject is a child under 8 years of age.

In various embodiments, the subject who is treated for HAEC has been diagnosed with HAEC. In other embodiments, the subject who is treated for HAEC is suspected to have HAEC. In still other embodiments, subject who is treated for HAEC is suspected to have a high quantity of a fungal species; for example in the gastrointestinal tract or in stool. For example, the high quantity of the fungal species can be quantified from a stool sample.

In various embodiments, the subject who is treated for HAEC has Hirschsprung disease (HSCR) or is suspected to have HSCR.

In various embodiments, the subject who is treated for HAEC has been diagnosed with HAEC according the various methods of the present invention. For example, the subject has been diagnosed with HAEC by quantitating the amount of a fungal species, for example, in a stool sample. Quantitative polymerase chain reaction (qPCR) detection of the amount of the fungal species can be used to diagnose the subject with HAEC.

In various embodiments, quantitating the amount of a fungal species in the biological sample can comprise using next generation sequencing (NGS).

In various embodiments, the subject for whom the likelihood of HAEC is reduced is suspected to have a higher likelihood of developing HAEC. In various embodiments, the subject for whom the likelihood of HAEC is reduced has Hirschsprung disease (HSCR) or is suspected to have HSCR.

In still other embodiments, the subject for whom the likelihood of HAEC is reduced is suspected to have a high quantity of a fungal species, for example in the gastrointestinal tract or in stool. For example, the high quantity of the fungal species can be quantified from a stool sample.

In various embodiments, the subject for whom the likelihood of HAEC is reduced has been determined to have a high quantity of a fungal species according the various methods of the present invention. For example, the subject has been determined to have a high quantity of a fungal species by quantitating the amount of a fungal species. Quantitative polymerase chain reaction (qPCR) detection of the amount of the fungal species can be used.

In various embodiments, quantitating the amount of a fungal species in the biological sample can comprise using next generation sequencing (NGS).

In various embodiments the subject who is susceptible of developing Hirschsprung-associated enterocolitis is a subject who has Hirschsprung disease (HSCR). In various embodiments, that subject is a child; for example under 18 years of age. In various embodiments, the younger the child, the higher the risk for developing HAEC. In various embodiments, the child is 13 years of age or less. In various embodiments, the subject is a child under 8 years of age.

### Fungal Species

In various embodiments, Hirschsprung-associated enterocolitis is caused, at least in part, by a Candida species. In various embodiments, Candida species is C. albicans or C. tropicalis.

In various embodiments, the Hirschsprung-associated enterocolitis is caused, at least in part, by a Saccharomyces species. In various embodiments, the Saccharomyces species is Saccharomyces cerevisiae.

These fungal species can also be the fungal species that are quantified to determine if a subject has HAEC, or is susceptible to HAEC.

### Antifungal Agents

In various embodiments, the antifungal agent is isavuconazole (e.g., isavuconazonium sulfate), posaconazole, itraconazole, efinaconazole, tavaborole, luliconazole, terbinafine, auriclosene, E-1224 (Eisai Co., Ltd.), VT-1161 (Viamet Pharmaceuticals, Inc.), NDV-3 (NovaDigm Therapeutics, Inc.), NDV-3A (NovaDigm Therapeutics, Inc.), SQ-109 (Sequella, Inc.), MGCD-290 (Mirati Therapeutics, Inc.), ME-1111 (Meiji Seika Pharma Co., Ltd.), LACTIN-V (Osel, Inc.), or combinations thereof, or salts thereof.

In various embodiments, the antifungal agent is natamycin, fluconazole or a combination thereof, or salts thereof.

In various embodiments, the antifungal agent is natamycin or a salt thereof. In other embodiments, the antifungal agent is fluconazole or a salt thereof.

### Antibiotic Agents

In various embodiments, the antibiotic is metronidazole, vancomycin, enrofloxacin, or a combination thereof.

In certain embodiments, the antibiotic is a nitroimidazole; for example, metronidazole. In certain embodiments, the antibiotic is a glycopeptide antibiotic; for example, vancomycin, tobramycin. In certain embodiments, the antibiotic is an aminoglycoside; for example, gentamicin. In certain embodiments, the antibiotic is a fluoroquinolone, for example, ciprofloxacin, levofloxacin. In certain embodiments, the antibiotic is a piperacillin and tazobactam combination (e.g., ZOSYN).

### Diagnosis and Selection of Treatment

Various embodiments provide for a method of diagnosing Hirschsprung-associated enterocolitis.

In various embodiments, the method comprises providing a biological sample; quantitating the amount of a fungal species in the biological sample; diagnosing Hirschsprung-associated enterocolitis when the quantity of the fungal species is greater than a reference value.

Various embodiments provide for a method of selecting a treatment for a subject suspected of having Hirschsprung-associated enterocolitis.

In various embodiments, the method comprises providing or obtaining a biological sample; quantitating the amount of a fungal species in the biological sample; and selecting an antifungal treatment for Hirschsprung-associated enterocolitis when the quantity of the fungal species is greater than a reference value.

In other embodiments, the method comprises providing or obtaining a biological sample; quantitating the amount of a fungal species in the biological sample; and selecting an antifungal treatment and an antibiotic for Hirschsprung-associated enterocolitis when the quantity of the fungal species is greater than a reference value.

Quantitative polymerase chain reaction (qPCR) detection of the amount of the fungal species can be used.

In various embodiments, quantitating the amount of a fungal species in the biological sample can comprise using next generation sequencing (NGS).

### Biological Sample

Examples of biological samples that can be used in accordance with various embodiments of the present invention include, but are not limited to mammalian body fluids, sera such as blood (including whole blood as well as its plasma and serum), CSF (spinal fluid), urine, gastric or intestinal fluids, sweat, saliva, tears, pulmonary secretions, breast aspirate, prostate fluid, seminal fluid, stool, cervical scraping, cysts, amniotic fluid, intraocular fluid, mucous, moisture in breath, animal tissue, cell lysates, tumor tissue, hair, skin, buccal scrapings, nails, bone marrow, cartilage, prions, bone powder, ear wax, etc. or even from external or archived sources (i.e., fresh, frozen or paraffin-embedded). Samples, such as body fluids or sera, obtained during the course of clinical trials may be particularly advantageous for use in connection with research, although samples obtained directly from living subjects under alternate conditions or for other purposes may be readily used as well. In particular embodiments, the biological sample is stool.

### Reference Values

The reference value can be calculated from a control group. In some embodiments, the control group is composed of healthy subjects who do not have Hirschsprung-associated enterocolitis. In some embodiments, the control group is composed of subjects who have Hirschsprung disease but do not have Hirschsprung-associated enterocolitis. In some embodiments, the control group is composed of subjects who do not have enterocolitis.

### Methods of Quantitating the Amount of a Fungal Species

In various embodiments, quantitating the amount of a fungal species in the biological sample comprises using quantitative polymerase chain reaction (qPCR) detection of the amount of the fungal species. In various embodiments, quantitating the amount of a fungal species in the biological sample can comprise using next generation sequencing (NGS).

In various embodiments, quantitating the amount of a fungal species in the biological sample comprises using quantitative polymerase chain reaction (qPCR) detection of the amount of the fungal species and the primers TTTATCAACTTGTCACACCAGA (SEQ ID NO:1) (Forward) and ATCCCGCCTTACCACTACCG (SEQ ID NO:2) (Reverse) are used for C. albicans, and the primers CAATCCTACCGCCAGAGGTTAT (SEQ ID NO:3) (Forward) and TGGCCACTAGCAAAATAAGCG (SEQ ID NO:4) (Reverse) are used for C. tropicalis. In further embodiments, quantitating the amount of a fungal species in the biological sample comprises using quantitative polymerase chain reaction (qPCR) detection of the amount of the fungal species and one or more primers having SEQ ID NO:1, SEQ ID NO:2, SEQ ID NO:3, SEQ ID NO:4, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, or SEQ ID NO:20. In various embodiments, two or more primers, four or more primers, six or more primers, eight or more primers, ten or more primers, twelve or more primers, fourteen or more primers or 16 or more primers are used. In various embodiments, 2, 4, 6, 8, 10, 12, 14, or 16 primers are used; for example, the primers from table 4 below.

TABLE 4
List of qPCR primers used for quantifying fungal
species

| | ID# | | SEQ |
|---|---|---|---|
| | in | qPCR Primer Sequence | ID |
| Fungal Species | Lab | (5′-3′) | NO: |
| Candida | Y1F | CCTGTTTGAGCGTCGTTT | 9 |
| | Y1R | TCCTCCGCTTATTGATAT | 10 |
| Candida albicans | Y2F | TTTATCAACTTGTCACACCAGA | 1 |
| (C. albicans) | Y2R | ATCCCGCCTTACCACTACCG | 2 |
| Candida | Y3F | AGATTAAACTCAACCAA | 11 |
| parapsilosis | Y3R | CCTATCCATTAGTTTATACTCCGC | 12 |
| (C. parapsilosis) | | | |
| Candida | Y4F | CAATCCTACCGCCAGAGGTTAT | 3 |
| tropicalis | Y4R | TGGCCACTAGCAAAATAAGCGT | 4 |
| (C. tropicalis) | | | |
| Saccharomyces | Y5F | AGGAGTGCGGTTCTTTG | 13 |
| cerevisiae | Y5R | TACTTACCGAGGCAAGCTACA | 14 |
| (S. cerevisiae) | | | |
| Fungal 18S rDNA | Y6F | ATTGGAGGGCAAGTCTGGTG | 15 |
| | Y6R | CCGATCCCTAGTCGGCATAG | 16 |
| Fungal IT S1-2 | Y7F | CTTGGTCATTTAGAGGAAGTAA | 17 |
| | Y7R | GCTGCGTTCTTCATCGATGC | 18 |
| Candida | Y8F | TTTGGTGGCGGGAGCAATCCT | 19 |
| tropicalis | Y8R | CGATGCGAGAACCAAGAGATCCGT | 20 |
| (C. tropicalis) | | | |

F = forward; R = reverse.

### Fungal Species

In various embodiments, the fungal species is Candida species. In various embodiments, Candida species is C. albicans or C. tropicalis.

In various embodiments, the fungal species is Saccharomyces species. In various embodiments, the Saccharomyces species is Saccharomyces cerevisiae.

### Antifungal Agents

In various embodiments, the antifungal agent is isavuconazonium sulfate, posaconazole, itraconazole, efinaconazole, tavaborole, luliconazole, terbinafine, auriclosene, E-1224 (Eisai Co., Ltd.), VT-1161 (Viamet Pharmaceuticals, Inc.), NDV-3 (NovaDigm Therapeutics, Inc.), NDV-3A (NovaDigm Therapeutics, Inc.), SQ-109 (Sequella, Inc.), MGCD-290 (Mirati Therapeutics, Inc.), ME-1111 (Meiji Seika Pharma Co., Ltd.), LACTIN-V (Osel, Inc.), or combinations thereof.

In various embodiments, the antifungal agent is natamycin, fluconazole or a combination thereof.

In various embodiments, the antifungal agent is natamycin or a salt thereof. In other embodiments, the antifungal agent is fluconazole or a salt thereof.

### Antibiotic Agents

In various embodiments, the antibiotic is metronidazole, vancomycin, enrofloxacin, or a combination thereof.

In certain embodiments, the antibiotic is a nitroimidazole; for example, metronidazole. In certain embodiments, the antibiotic is a glycopeptide antibiotic; for example, vancomycin, tobramycin. In certain embodiments, the antibiotic is an aminoglycoside; for example, gentamicin. In certain embodiments, the antibiotic is a fluoroquinolone, for example, ciprofloxacin, levofloxacin. In certain embodiments, the antibiotic is a piperacillin and tazobactam combination (e.g., ZOSYN).

### Pharmaceutical Compositions

In various embodiments, the present invention provides pharmaceutical compositions including a pharmaceutically acceptable excipient along with a therapeutically effective amount of an antifungal. “Pharmaceutically acceptable excipient” means an excipient that is useful in preparing a pharmaceutical composition that is generally safe, non-toxic, and desirable, and includes excipients that are acceptable for veterinary use as well as for human pharmaceutical use. Such excipients may be solid, liquid, semisolid, or, in the case of an aerosol composition, gaseous.

In various embodiments, the pharmaceutical compositions according to the invention may be formulated for delivery via any route of administration. “Route of administration” may refer to any administration pathway known in the art, including but not limited to aerosol, nasal, oral, transmucosal, transdermal or parenteral. “Transdermal” administration may be accomplished using a topical cream or ointment or by means of a transdermal patch. “Parenteral” refers to a route of administration that is generally associated with injection, including intraorbital, infusion, intraarterial, intracapsular, intracardiac, intradermal, intramuscular, intraperitoneal, intrapulmonary, intraspinal, intrasternal, intrathecal, intrauterine, intravenous, subarachnoid, subcapsular, subcutaneous, transmucosal, or transtracheal. Via the parenteral route, the compositions may be in the form of solutions or suspensions for infusion or for injection, or as lyophilized powders. Via the enteral route, the pharmaceutical compositions can be in the form of tablets, gel capsules, sugar-coated tablets, syrups, suspensions, solutions, powders, granules, emulsions, microspheres or nanospheres or lipid vesicles or polymer vesicles allowing controlled release. Via the parenteral route, the compositions may be in the form of solutions or suspensions for infusion or for injection. Via the topical route, the pharmaceutical compositions based on compounds according to the invention may be formulated for treating the skin and mucous membranes and are in the form of ointments, creams, milks, salves, powders, impregnated pads, solutions, gels, sprays, lotions or suspensions. They can also be in the form of microspheres or nanospheres or lipid vesicles or polymer vesicles or polymer patches and hydrogels allowing controlled release. These topical-route compositions can be either in anhydrous form or in aqueous form depending on the clinical indication. Via the ocular route, they may be in the form of eye drops.

The pharmaceutical compositions according to the invention can also contain any pharmaceutically acceptable carrier. “Pharmaceutically acceptable carrier” as used herein refers to a pharmaceutically acceptable material, composition, or vehicle that is involved in carrying or transporting a compound of interest from one tissue, organ, or portion of the body to another tissue, organ, or portion of the body. For example, the carrier may be a liquid or solid filler, diluent, excipient, solvent, or encapsulating material, or a combination thereof. Each component of the carrier must be “pharmaceutically acceptable” in that it must be compatible with the other ingredients of the formulation. It must also be suitable for use in contact with any tissues or organs with which it may come in contact, meaning that it must not carry a risk of toxicity, irritation, allergic response, immunogenicity, or any other complication that excessively outweighs its therapeutic benefits.

The pharmaceutical compositions according to the invention can also be encapsulated, tableted or prepared in an emulsion or syrup for oral administration. Pharmaceutically acceptable solid or liquid carriers may be added to enhance or stabilize the composition, or to facilitate preparation of the composition. Liquid carriers include syrup, peanut oil, olive oil, glycerin, saline, alcohols and water. Solid carriers include starch, lactose, calcium sulfate, dihydrate, terra alba, magnesium stearate or stearic acid, talc, pectin, acacia, agar or gelatin. The carrier may also include a sustained release material such as glyceryl monostearate or glyceryl distearate, alone or with a wax.

The pharmaceutical preparations are made following the conventional techniques of pharmacy involving milling, mixing, granulation, and compressing, when necessary, for tablet forms; or milling, mixing and filling for hard gelatin capsule forms. When a liquid carrier is used, the preparation will be in the form of a syrup, elixir, emulsion or an aqueous or non-aqueous suspension. Such a liquid formulation may be administered directly p.o. or filled into a soft gelatin capsule.

The pharmaceutical compositions according to the invention may be delivered in a therapeutically effective amount. The precise therapeutically effective amount is that amount of the composition that will yield the most effective results in terms of efficacy of treatment in a given subject. This amount will vary depending upon a variety of factors, including but not limited to the characteristics of the therapeutic compound (including activity, pharmacokinetics, pharmacodynamics, and bioavailability), the physiological condition of the subject (including age, sex, disease type and stage, general physical condition, responsiveness to a given dosage, and type of medication), the nature of the pharmaceutically acceptable carrier or carriers in the formulation, and the route of administration. One skilled in the clinical and pharmacological arts will be able to determine a therapeutically effective amount through routine experimentation, for instance, by monitoring a subject's response to administration of a compound and adjusting the dosage accordingly. For additional guidance, see Remington: The Science and Practice of Pharmacy (Gennaro ed. 20th edition, Williams & Wilkins PA, USA) (2000).

Typical dosages of an effective antifungal or antibiotic can be in the ranges recommended by the manufacturer where known therapeutic compounds are used, and also as indicated to the skilled artisan by the in vitro responses or responses in animal models. Such dosages typically can be reduced by up to about one order of magnitude in concentration or amount without losing the relevant biological activity. Thus, the actual dosage will depend upon the judgment of the physician, the condition of the patient, and the effectiveness of the therapeutic method based, for example, on the in vitro responsiveness of the relevant primary cultured cells or histocultured tissue sample, or the responses observed in the appropriate animal models, as previously described.

In some embodiments, dosages of the fluconazole can be about 6-12 milligram/kg/day; dosages of natamycin can be 25-250 microgram/kg/day; dosages of metronidazole can be 7.5-15 mg/kg IV/PO q6 h; dosages of vancomycin can be 10-15 mg/kg IV q6h, 40 mg/kg up to 2000 mg/day PO, dosages of gentamicin can be 6-7.5 mg/kg/day divided q8 h; dosages of tobramycin can be 6-7.5 mg/kg/day divided q8 h; dosages of ciprofloxacin can be 200-400 mg IV q8-12 h, PO 250-750 mg q12 h; dosages of levofloxacin can be 250-500 mg IV/PO q12 h; and dosages of ZOSYN can be 3.375 g IV q4-6 h.

As used herein, “pharmaceutically acceptable salts or prodrugs” are salts or prodrugs that are, within the scope of sound medical judgment, suitable for use in contact with the tissues of subject without undue toxicity, irritation, allergic response, and the like, commensurate with a reasonable benefit/risk ratio, and effective for their intended use.

As used herein, a prodrug is a compound that, upon in vivo administration, is metabolized or otherwise converted to the biologically, pharmaceutically or therapeutically active form of the compound. A prodrug of the one or more compounds as disclosed herein or a mutant, variant, analog or derivative thereof can be designed to alter the metabolic stability or the transport characteristics of one or more compounds as disclosed herein or a mutant, variant, analog or derivative thereof, to mask side effects or toxicity, to improve the flavor of a compound or to alter other characteristics or properties of a compound. By virtue of knowledge of pharmacodynamic processes and drug metabolism in vivo, once a pharmaceutically active form of the one or more compounds as disclosed herein or a mutant, variant, analog or derivative thereof, those of skill in the pharmaceutical art generally can design prodrugs of the compound. Suitable examples of prodrugs include methyl, ethyl and glycerol esters of the corresponding acid.

### Kits

The present invention is also directed to kits to treat Hirschsprung-associated enterocolitis, kits to reduce the likelihood of having Hirschsprung-associated enterocolitis, kits to diagnose Hirschsprung-associated enterocolitis, and kits to select a treatment for Hirschsprung-associated enterocolitis. The kit is useful for practicing the inventive method of treating HAEC, reducing the likelihood of having HAEC, diagnosing HAEC, or selecting a treatment for HAEC. The kit is an assemblage of materials or components, including at least one of the inventive compositions. Thus, in some embodiments the kit contains a composition including antifungals, or primers as described above.

The exact nature of the components configured in the inventive kit depends on its intended purpose. For example, some embodiments are configured for the purpose of treating HAEC. Other embodiments are configured for the purpose of diagnosing HAEC. In one embodiment, the kit is configured particularly for the purpose of treating mammalian subjects. In another embodiment, the kit is configured particularly for the purpose of treating human subjects. In another embodiment, the kit is configured particularly for the purpose of treating human children. In further embodiments, the kit is configured for veterinary applications, treating subjects such as, but not limited to, farm animals, domestic animals, and laboratory animals.

Instructions for use may be included in the kit. “Instructions for use” typically include a tangible expression describing the technique to be employed in using the components of the kit to effect a desired outcome, such as to treat HAEC. Optionally, the kit also contains other useful components, such as, diluents, buffers, pharmaceutically acceptable carriers, syringes, catheters, applicators, pipetting or measuring tools, bandaging materials or other useful paraphernalia as will be readily recognized by those of skill in the art.

The materials or components assembled in the kit can be provided to the practitioner stored in any convenient and suitable ways that preserve their operability and utility. For example the components can be in dissolved, dehydrated, or lyophilized form; they can be provided at room, refrigerated or frozen temperatures. The components are typically contained in suitable packaging material(s). As employed herein, the phrase “packaging material” refers to one or more physical structures used to house the contents of the kit, such as inventive compositions and the like. The packaging material is constructed by well-known methods, preferably to provide a sterile, contaminant-free environment. As used herein, the term “package” refers to a suitable solid matrix or material such as glass, plastic, paper, foil, and the like, capable of holding the individual kit components. Thus, for example, a package can be a glass vial used to contain suitable quantities of an inventive composition containing an antifungal, antibiotic, and/or primers. The packaging material generally has an external label which indicates the contents and/or purpose of the kit and/or its components.

### Computer Implementation Systems and Methods

In certain embodiments, the methods of the invention implement a computer program to calculate fungal quantity. For example, a computer program can be used to perform the algorithms described herein. A computer system can also store and manipulate data generated by the methods of the present invention which comprises a plurality of hybridization signal changes/profiles during approach to equilibrium in different hybridization measurements and which can be used by a computer system in implementing the methods of this invention. In certain embodiments, a computer system receives probe hybridization data; (ii) stores probe hybridization data; and (iii) compares probe hybridization data to determine the quantity of the fungal species. Whether the quantity is higher or lower than the reference value is calculated. In some embodiments, a computer system (i) compares the fungal quantity to a threshold value or reference value; and (ii) outputs an indication of whether said fungal quantity is above or below a threshold or reference value, or the presence of a disease or condition based on said indication. In certain embodiments, such computer systems are also considered part of the present invention.

Numerous types of computer systems can be used to implement the analytic methods of this invention according to knowledge possessed by a skilled artisan in the bioinformatics and/or computer arts.

Several software components can be loaded into memory during operation of such a computer system. The software components can comprise both software components that are standard in the art and components that are special to the present invention (e.g., dCHIP software described in Lin et al. (2004) Bioinformatics 20, 1233-1240; CRLMM software described in Silver et al. (2007) Cell 128, 991-1002; Aroma Affymetrix software described in Richardson et al. (2006) Cancer Cell 9, 121-132. The methods of the invention can also be programmed or modeled in mathematical software packages that allow symbolic entry of equations and high-level specification of processing, including specific algorithms to be used, thereby freeing a user of the need to procedurally program individual equations and algorithms. Such packages include, e.g., Matlab from Mathworks (Natick, Mass.), Mathematica from Wolfram Research (Champaign, Ill.) or S-Plus from MathSoft (Seattle, Wash.). In certain embodiments, the computer comprises a database for storage of hybridization signal profiles. Such stored profiles can be accessed and used to calculate a fungal quantity.

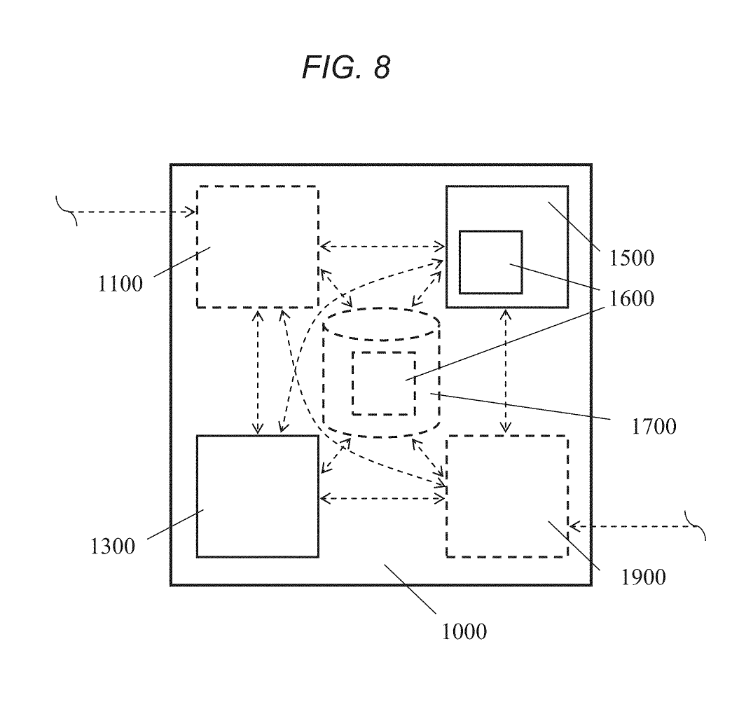

In a non-limiting example, FIG. 8 depicts a device or a computer system 1000 comprising one or more processors 1300 and a memory 1500 storing one or more programs 1600 for execution by the one or more processors 1300.

In some embodiments, the device or computer system 1000 can further comprise a non-transitory computer-readable storage medium 1700 storing the one or more programs 1600 for execution by the one or more processors 1300 of the device or computer system 1000.

In some embodiments, the device or computer system 1000 can further comprise one or more input devices 1100, which can be configured to send or receive information to or from any one from the group consisting of: an external device (not shown), the one or more processors 1300, the memory 1500, the non-transitory computer-readable storage medium 1700, and one or more output devices 1900.

In some embodiments, the device or computer system 1000 can further comprise one or more output devices 1900, which can be configured to send or receive information to or from any one from the group consisting of: an external device (not shown), the one or more processors 1300, the memory 1500, and the non-transitory computer-readable storage medium 1700.

Each of the above identified modules or programs corresponds to a set of instructions for performing a function as described by the present invention. These modules and programs (i.e., sets of instructions) need not be implemented as separate software programs, procedures or modules, and thus various subsets of these modules may be combined or otherwise re-arranged in various embodiments. In some embodiments, memory may store a subset of the modules and data structures identified above. Furthermore, memory may store additional modules and data structures not described above.

The illustrated aspects of the disclosure may also be practiced in distributed computing environments where certain tasks are performed by remote processing devices that are linked through a communications network. In a distributed computing environment, program modules can be located in both local and remote memory storage devices.

Moreover, it is to be appreciated that various components described herein can include electrical circuit(s) that can include components and circuitry elements of suitable value in order to implement the embodiments of the subject innovation(s). Furthermore, it can be appreciated that many of the various components can be implemented on one or more integrated circuit (IC) chips. For example, in one embodiment, a set of components can be implemented in a single IC chip. In other embodiments, one or more of respective components are fabricated or implemented on separate IC chips.

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

### Example 1

### Patients

Children with Hirschsprung disease who met inclusion criteria were enrolled from four member institutions of the HAEC Collaborative Research Group (HCRG): Cedars-Sinai Medical Center, Los Angeles, Calif.; Astrid Lindgren Children's Hospital, Karolinska University Hospital, Stockholm, Sweden; Children's Hospital Los Angeles, Los Angeles, Calif.; Children's Hospital of Oakland, Oakland, Calif. Written informed consent was obtained from a parent by the attending surgeons or research nurses at each site. We enrolled 20 children with HSCR, 10 never had a history of enterocolitis and 10 had a history of at least one episode of HAEC based on HAEC scoring system described by Pastor et al. (Development of a standardized definition for Hirschsprung's-associated enterocolitis: a Delphi analysis. J PEDIATR SURG 2009. 44: 251-256.) Detailed clinical information was collected using standardized questionnaires that included demographic, medical history, surgical history, radiographic, pathological, diet, medications (including antibiotics), probiotics and complications. The HCRG data was stored in a secure SQL relational database at the data coordinating center at CSMC.

### Patients Excluded From Microbiome Analysis

Two subjects were excluded from analysis: subject 03-0003 was excluded because he had a diverting ileostomy (that is, the patient had fecal stream diversion after pull-through); and 02-0039 was excluded because he had an active HAEC episode at the time of stool collection. Subject 04-0005 was excluded from fungal microbiome analysis only due to failure to pass quality control after sequencing, although bacterial microbiome analysis was completed.

### Fecal DNA Isolation

Within one week of enrollment in the study, stool was collected and immediately frozen at −80° C. All samples were kept frozen and shipped to the coordinating site (CSMC) where all of the samples were prepared for bacterial and fungal DNA analysis. (Iliev et al., Interactions between commensal fungi and the C-type lectin receptor Dectin-1 influence colitis. SCIENCE 2012. 336: 1314-1317) Fecal samples were thawed and resuspended in 50 mM Tris buffer (pH7.5) containing 1 mM EDTA, 0.2% β-mercaptoethanol (Sigma) and 1000 U/ml of lyticase (Sigma). The mix was incubated at 37° C. for 30 min and fungal genomic DNA was isolated by using QIAamp DNA Stool Mini Kit (Qiagen) according to the manufacturer's instructions.

### Bacterial and Fungal Amplicon Preparation

Bacterial 16S rRNA gene amplicons spanning variable regions one to four (V1-4) were generated in 20 mL PCR reactions using 20 ng of fecal DNA with 25 cycles using high-fidelity Phusion Polymerase (New England Biolabs, Beverly, Mass.) at 52.7° C. annealing using with degenerate 8F (AGAGTTTGATCMTGGCTCAG (SEQ ID NO:5)) and R357 (CTGCTGCCTYCCGTA (SEQ ID NO:6)) primers. Fungal ITS-1 amplicons were generated in 20 mL PCR reactions using 20 ng of fecal DNA with 35 cycles using Phusion Polymerase at 56.1° C. annealing using ITS1F (CTTGGTCATTTAGAGGAAGTAA (SEQ ID NO:7)) and ITS2R (GCTGCGTTCTTCATCGATGC (SEQ ID NO:8) primers yielded sufficient amplification of ITS targets. All PCR reactions were purified using Agencourt AmPure Magnetic Beads (Beckman), resuspended in 20 mL of nuclease-free water and quantified using a Qubit fluorometer (Invitrogen, Carlsbad, Calif.).

### Library Preparation of Bacterial and Fungal Libraries

Paired-end adapters with unique indexes were ligated to 100 ng of 16S amplicons and used to generate Ion Torrent sequencing libraries using the Ion Xpress Library Kit (Life Technologies, Carlsbad, Calif.). Illumina paired-end adapters with unique indexes were ligated to 100 ng of ITS-1 amplicons using a modified TruSeq DNA Sample Preparation (Illumina, San Diego, Calif.) where adapters and PCR primers were diluted 1:10 to accommodate lower input of amplicon mass for both 16S and ITS-1 preparations. Library enrichment was performed with 10 cycles of PCR and purified using Agencourt Ampure Magnetic Beads (Beckman). All libraries were subjected to quality control using qPCR, DNA 1000 Bioanalyzer (Agilent), and Qubit (Life Technologies, Carlsbad, Calif.) to validate and quantitate library construction then pooled at equimolar concentrations.

### Reference Strains and Culture Conditions

Fungal reference strains Candida albicans (ATCC 90028) and Candida tropicalis (ATCC 750) were obtained from the American Type Culture Collection (Manassas, Va.). Fungi were cultured in aerobic conditions on Sabouraud Dextrose Broth (SDB; EMD Chemicals) for overnight at 37° C. The cultured cells were harvested for DNA preparation using the QIAamp DNA Stool Mini Kit (Qiagen, Inc., USA).

### Library Sequencing

Pooled libraries were assayed on Agilent Bioanalyzer (Santa Clara, Calif.) to check final sizing and to check for small fragments as well as KAPA Biosciences qPCR for quantitation. 16S samples were multiplexed and sequenced on the Ion Torrent PGM on a 318 chip with 400 bp chemistry. For ITS-1 sequencing final diluted pool was amplified on to a Single End flow cell using clonal bridge amplification on the MiSeq. 250 single-end sequencing-by-synthesis was performed using the MiSeq lumina sequencer (Illumina, San Diego, Calif.).

### Next Generation Sequencing (NGS) Data Analysis and Species Identification.

Bacterial Species. Ion Torrent reads shorter than 200 bp, or not containing the designed 16S primers (>2nt mismatches) were discarded. 300 bp sequences of remaining high-quality reads were aligned to the Greengenes reference database (February 2011 release) using BLAST v2.2.22 in QIIME v1.5 wrapper (Caporaso et al., QIIME allows analysis of high-throughput community sequencing data. NAT METHODS 2010. 7: 335-336) with an identity percentage ≧97% to select the operational taxonomic units (OTUs). Taxonomy for each sequence was assigned using the Ribosomal Database Project (RDP) classifier v2.2.

### Fungal Species.

FASTQ data was de-multiplexed and filtered through a stringent quality control procedure to ensure that only high-quality sequences were analyzed further. To identify fungal species, the filtered reads were aligned with the Findley ITS Database (Findley et al., Topographic diversity of fungal and bacterial communities in human skin. NATURE 2013. 498: 367-370) using BLAST v2.2.22 in QIIME v1.5.0 wrapper (Caporaso et al.) with an identity percentage ≧97% for OUT picking. Chosen OTUs were compiled into genera or families.

### Diversity Indices

The original OTU table was randomly subsampled (rarefied) to create a series of subsampled OTU tables. Alpha diversity was calculated in QIIME on each sample using the OTU table and Shannon indices were collated into a single file and the number of species identified for both bacteria and fungi for each sample versus the depth of subsampling was plotted.

### Fungal Quantitative PCR

Fungal reference strain Candida albicans (ATCC 90028) was obtained from the American Type Culture Collection (Manassas, Va.). Fungi were cultured in aerobic conditions on Sabouraud Dextrose Broth (SDB; EMD Chemicals) for overnight at 37° C. The cultured cells were harvested for DNA preparation using the QIAmp DNA Stool Mini Kit (Qiagen, Inc., USA).

Quantitative PCR was performed on DNA isolated from human stool using SYBR Green Kit (Bio-Rad). Specific primer pairs for Candida albicans and Candida tropicalis TTTATCAACTTGTCACACCAGA (SEQ ID NO:1) (Forward) and ATCCCGCCTTACCACTACCG (SEQ ID NO:2) (Reverse); and CAATCCTACCGCCAGAGGTTAT (SEQ ID NO:3) (Forward) and TGGCCACTAGCAAAATAAGCG (SEQ ID NO:4) (Reverse), respectively. (Hsu et al., Species identification of medically important fungi by use of real-time Light Cycler PCR. J MED MICROBIOL 2003. 52: 1071-1076) In a 20 ul of qPCR reactive mixture contained 2 ul of stool DNA (2˜100 ng), 10 ul of iQ SYBR Green Supermix (2×), 4 ul of forward primer (3 pmol/mL), and 4 ul of reverse primer (3 pmol/mL). The PCR protocol was modified from Iliev et al. (Interactions between commensal fungi and the C-type lectin receptor Dectin-1 influence colitis. SCIENCE 2012. 336: 1314-1317): Initial denaturation at 94° C. for 10 min, followed by 35 cycles of denaturation at 94° C. for 30 s, annealing at 55.3° C. for 30 s, and elongation at 72° C. for 2 min, followed by an elongation step at 72° C. for 30 min. C. albicans in fecal specimens was determined by using a standard curve generated by Candida albicans (ATCC 90028) DNA with 10-fold serial dilution from 10² ng to 10⁻⁴ ng against the threshold cycle C(t), and normalized to the amount of total fecal DNA being used. The qPCR results are reported as C. albicans cell numbers per ng fecal DNA, in which the C. albicans cell numbers were calculated from cell counts per nanogram (ng) DNA of reference Candida albicans (ATCC 90028) where 3×10⁻⁴ng DNA represents one cell.

### Patient Characteristics

Each group of subjects meeting inclusion criteria consisted of 8 males and 1 female. The median age of all children was 2.7 years (range 5 months to 8 years); the median age of the HSCR group was 2.3 years and the HAEC group was 3.5 years. Tables 1 and 2. Most subjects had aganglionic transition zones in the rectosigmoid colon region; one in the HSCR group had a transverse colon transition zone and one in the HAEC group had an ileal transition zone (total colonic aganglionosis). There were no significant differences in diet (breast milk vs. formula) or probiotic use in the children who developed HAEC compared with those who did not develop HAEC. Three children in the HAEC group received antibiotics within 2 months prior to stool collection: two for treatment of HAEC and one as daily prophylaxis for sickle cell disease, while none of the HSCR group received antibiotics. Three of the patients in the HAEC group developed HAEC as a complication within the first 30 days after pull-through procedure, while none of the HSCR patients had complications. One patient in the HSCR group had trisomy 21, while two patients in the HAEC cohort had trisomy 21 and one had sickle cell disease.

### Bacterial Microbiome Analysis

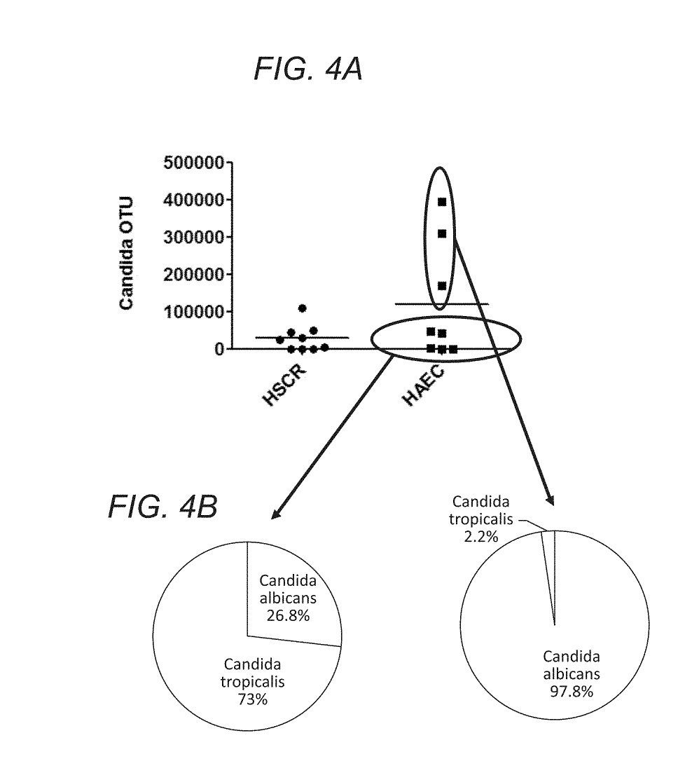

A mean of 16,304 sequences per sample were analyzed and an estimate of diversity in each group using rarefaction curves suggested greater diversity of bacterial species in the HAEC group compared with the HSCR patients (FIG. 5).

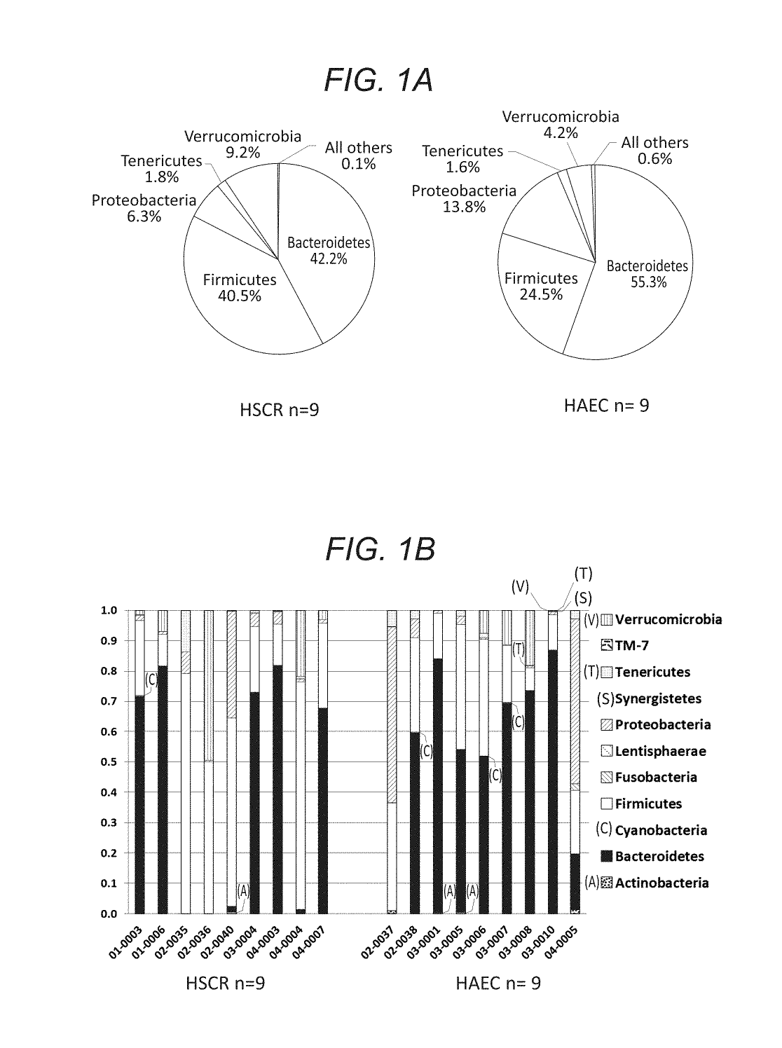

We next analyzed differences in the proportion of bacterial groups at the phylum level. Five phyla (Firmicutes, Bacteroidetes, Proteobacteria, Verrucomicrobia and Tenericutes) dominated the bacterial microbiota in most samples. FIG. 1A. The proportion of Firmicutes and Verrucomicrobia was lower in HAEC patients than in HSCR. We observed a lower proportion of Firmicutes and Verrucomicrobia, at 24.5% and 4.2% and a relative increased proportion of Bacteroidetes and Proteobacteria, at 55.3% and 13.8%, respectively in the HAEC group, when compared with the HSCR group of 40.5% and 9.2% for Firmicutes and Verrucomicrobia, respectively, and 42.2% and 6.3% for Bacteroidetes and Proteobacteria, respectively. Statistical comparisons for each phylum were performed and did not reach significance. The taxonomic composition of 11 phyla for each patient was performed and is shown in FIG. 1B. There appears to be clustering of composition by study site as noted in 02-0035, 02-0036, 02-0040 and 02-0037 from the Swedish cohort showing a reduction in proportion of Bacteroidetes, which is of unclear significance.

We further analyzed the differences in proportion at the genus level and similarly found no statistically significant differences between the groups.

### Fungal Microbiome Analysis

Heretofore, nothing is known about what commensal fungi populate the gut of children with HSCR or how they might contribute to HAEC. A mean of 168,805 sequences were generated were generated per patient and an estimate of diversity in each group using rarefaction curves suggested reduced diversity of fungal species in the HAEC compared with the HSCR patients (FIG. 5).

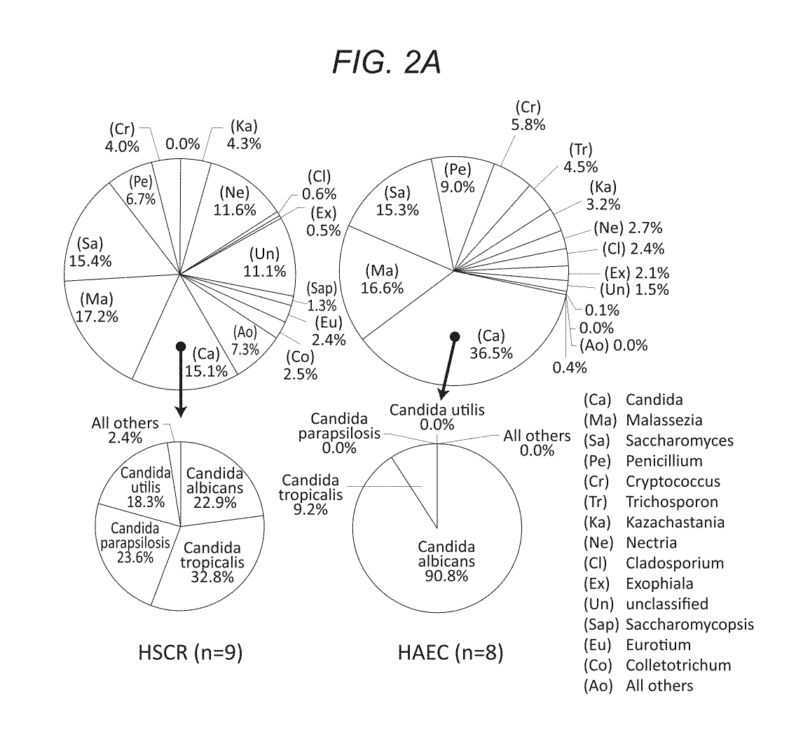

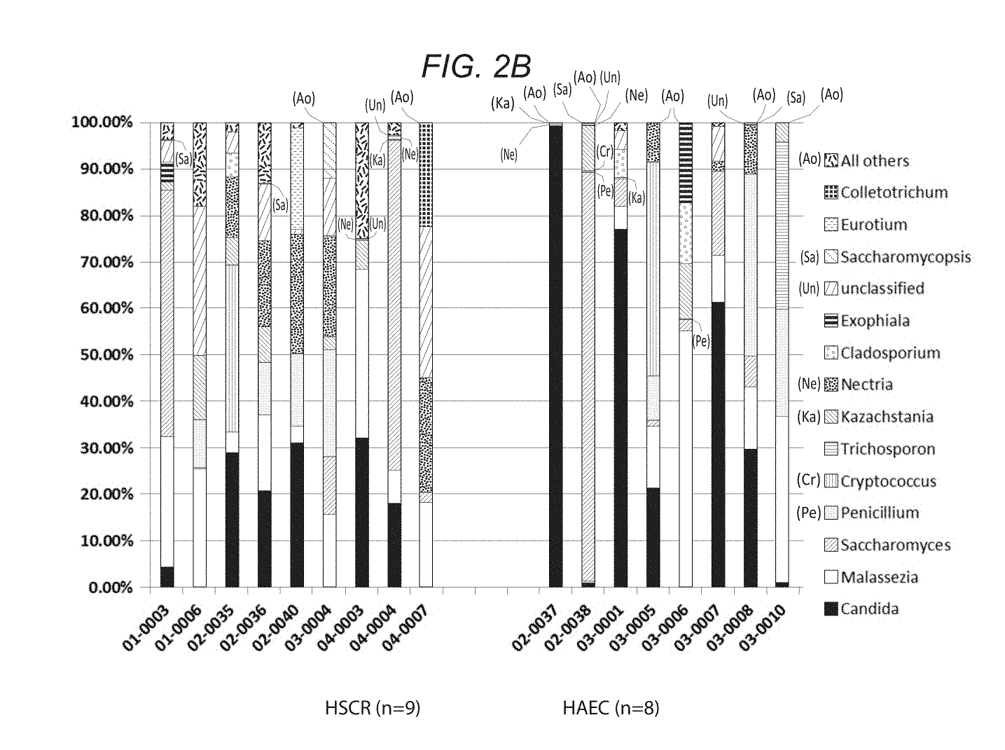

Detailed analysis identified 74 different well-annotated fungal genera, which illustrated the fungal diversity. Note that 89-98% of all fungal sequences identified belonged to 11 fungal genera in the samples analyzed. In HSCR group, we found that 15.1% of the sequences belong to Candida, while the HAEC group had a considerably larger portion at 36.5% of sequences, FIG. 2A (upper pie chart). The Candida species of the HSCR group was split between C. albicans, 22.9%; C. tropicalis 32.8%; C. parapsilosis 23.6%; C. utilis 18.3% while the HAEC patients had an overwhelming majority of C. albicans 90.8% and low C. tropicalis 9.2%, such that 33% of sequences belong to C. albicans. FIG. 2A (lower pie chart). The taxonomic composition of the 13 most abundant fungal genera for each subject was performed and is shown in FIG. 2B, and Candida was more abundant in the majority of the HAEC patients, but not all. To confirm the dramatically increased C. albicans observed in the HAEC group, quantitative PCR was performed for C. albicans in the same specimens and shown in FIG. 3. Three of eight HAEC patients showed especially elevated C. albicans, while only 1 of 9 HSCR patients showed elevated C. albicans. Subject 03-0010 had such low quantities of C. albicans DNA in the sample that no amplification was detected.

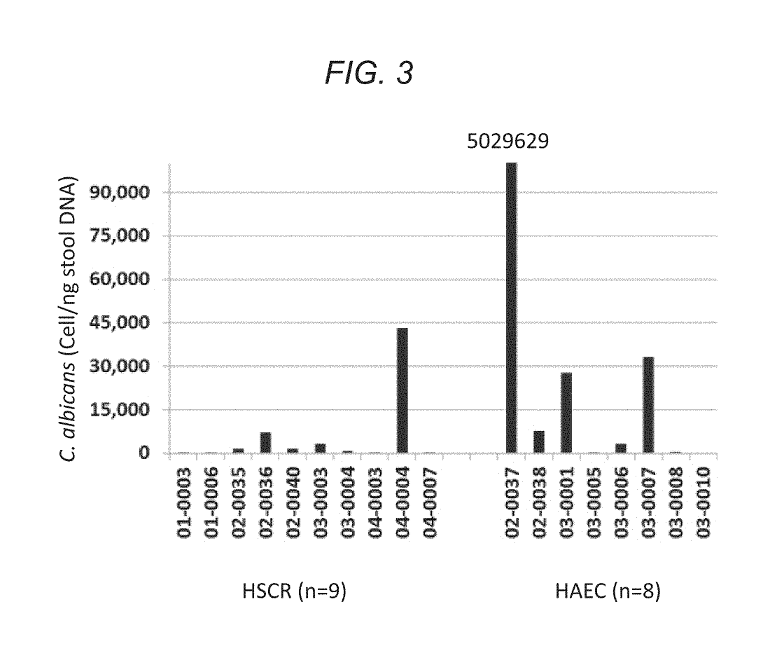

When the HSCR and HAEC groups were further analyzed for Candida OTU abundance, the identical three HAEC patients showed a high burden of Candida compared with the other HAEC and HSCR patients (FIG. 4A). There were statistically significant differences in Candida OTU abundance between both the HAEC “high burden” (285900±64620) and HSCR (37550±11210) (P<0.0001) and HAEC “low burden” patients (18910±10830) (P=0.001), respectively, but not between HSCR and HAEC “low burden” patients.

In the HAEC group, the species composition of “high burden” patients showed 97.8% was C. albicans and only 2.2% C. tropicalis compared with “low burden” patients 26.8% C. albicans and 73% C. tropicalis (FIG. 4B). Interestingly even the low burden HAEC group did have altered Candida community structure with just two species compared to more diverse Candida populations in the HSCR patients. Without being bound to any particular theory, this was the first study to identify Candida spp. as potentially playing a role in HAEC either as expanded commensal species as a consequence of enterocolitis (or treatment), or possibly as pathobioants contributing to the pathogenesis of HAEC.

Without being bound to any particular theory, the data suggest a model in which patients with genetic susceptibility to developing HAEC respond inappropriately to changes in the microbiota (with a particular interest in fungi) that are associated with care of Hirschsprung patients leading to a sustained and difficult-to-treat colitis and may lead to methods for early identification of patients at high risk of developing HAEC.

### Example 2

We have developed a rapid molecular method of quantitative real-time polymerase chain reaction (qPCR) to detect Candida albicans in human stools. To further evaluate our qPCR method as a rapid and potentially less expensive alternative to assessing C. albicans fecal load (and potentially other clinically relevant fungi), we compared it to the currently accepted culture-based method using the services off the Clinical Microbiology lab here at CSMC using 13 human stool specimens from HD patients with or without enterocolitis (HAEC). The comparison results are reported as below.

TABLE 3

| | | Culture results | qPCR results of C. albicans |
|---|---|---|---|
| | Subject | of C. albicans | (Number of |
| | ID# | (CFU/100 mg) | C albicans/100 mg stool) |
| 1 | 03-0001 | >500 | 588 |
| 2 | 03-0002 | 225* | 9402097* |
| 3 | 03-0004 | 0 | 3 |
| 4 | 03-0005 | 5 | 2 |
| 5 | 03-0006 | 0 | 6 |
| 6 | 03-0008 | 0 | 0 |
| 7 | 03-0009 | 200 | 3 |
| 8 | 03-0011 | 0 | 0 |
| 9 | 03-0012 | 0 | 3 |
| 10 | 03-0013 | 25 | 0 |
| 11 | 03-0018 | 0 | 1 |
| 12 | 03-0019 | 0 | 0 |
| 13 | 02-00037 | >10,000,000 | 25402166 |

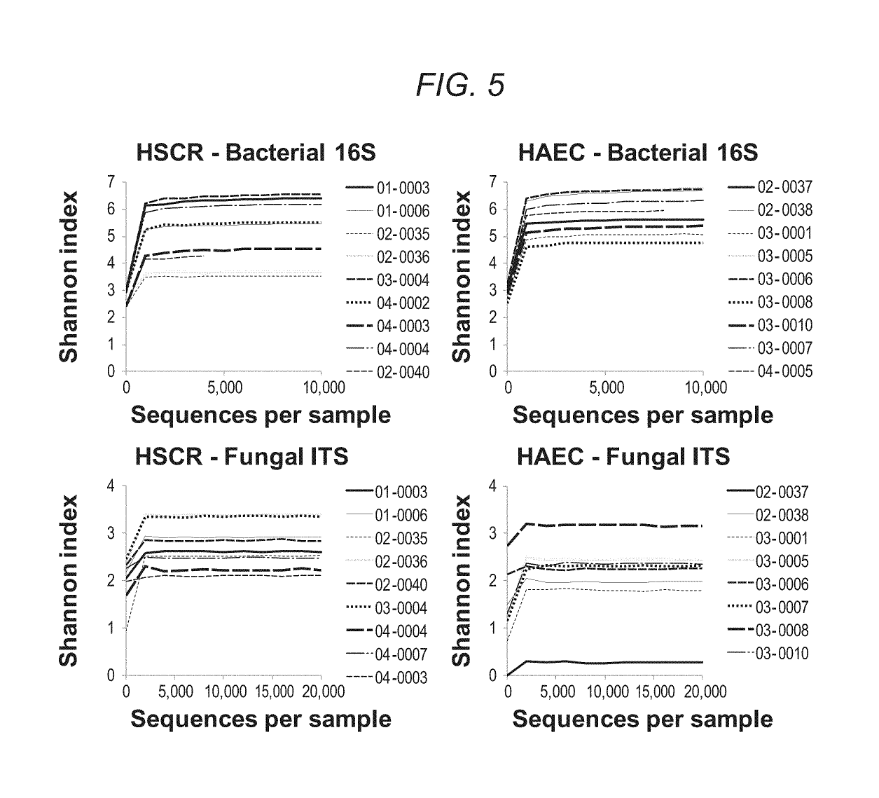

The Correlation analysis (Pearson) indicated that correlation coefficients r=0.935 (FIG. 6).

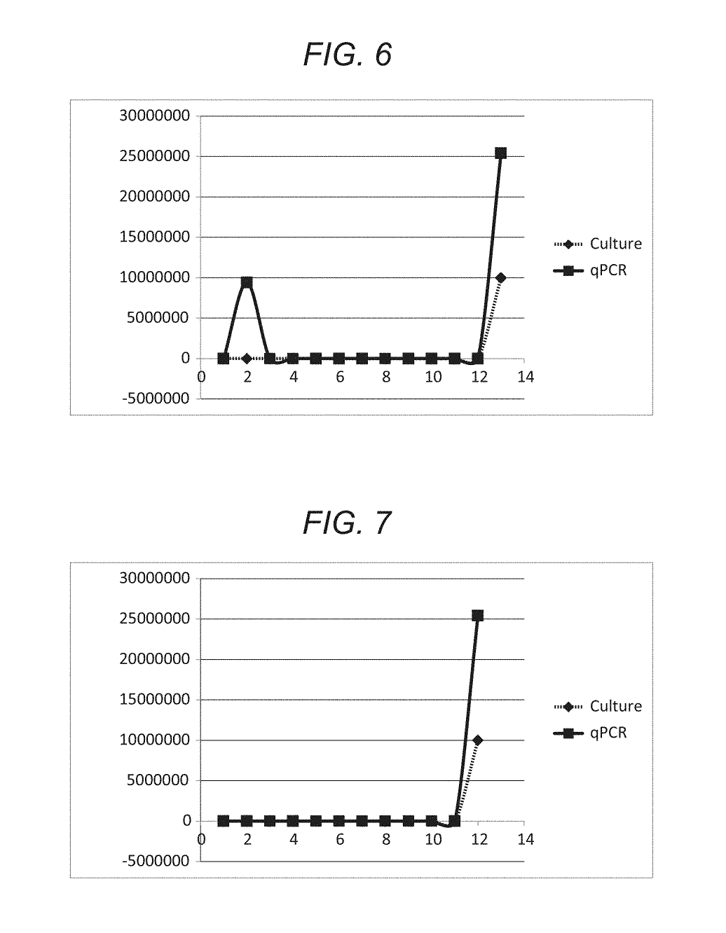

As qPCR result of patient 03-0002 was order of magnitudes larger than the result from standard culture, Further analysis was done by excluding this specimen and the Correlation coefficients r=1.0 .(Figure 7)

### Example 3

### Antibiotic and Antifungal Treatment in Murine Model of HAEC

We test antibiotic and antifungal agents and their combinations on the 129sv/Ednrb mouse strain shown to be a model of HAEC. The experimental groups and protocol are described below:

Medicated water which contains enrofloxacin (Baytril), metronidazole, vancomycin, natamycin (Pimaricin), fluconazole, and/or CRESEMBA (Isavuconazole sulfate) are administered to pregnant mice from the time that the pregnancy is noted, through weaning of the pups. In other words, the administration of medicated water to moms are stopped after weaning, and then continued with the weaned pups.

Six groups of animals (129sv/Ednrb-null mouse strain, n=5-8 per group) with different treatment are described as below. The dosage was calculated by the concentration of antibiotic and antifungal agents and assuming a mouse taking 5 ml water per day based on published data.

Group 1. Antibiotic water (ABX): Baytril (enrofloxacin) 0.25 mg/mL×5 mL water intake/day=1.25 mg/25 g mouse/day=50 mg/kg/day; Metronidazole 0.2 mg/mL×5 mL/day=1 mg/25 g mouse/day=40 mg/kg/day; Vancomycin 0.25 mg/mL×5 mL/day=1.25 mg/25 g mouse/day=50 mg/kg/day

Group 2. Antifungal agent 1—Natamycin (Pimaricin) water (AFX-1): Natamycin (Pimaricin) 1.25 ug/ml×5 mL water intake/day=6.25 ug/25 g mouse/day=250 ug/kg/day

Group 3. Antifungal agent 2—Fluconazole water (AFX-2): Fluconazole 0.5 mg/ml×5 mL water intake/day=2.5 mg/25 g mouse/day=100 mg/kg/day

Group 4. Antifungal agent 3—CRESEMBA water (AFX-3): CRESEMBA (isavuconazonium sulfate): 1 mg/ml×5 mL water intake/day=5 mg/25 g mouse/day=200 mg/kg/day

Group 5. Antibiotics plus Antifungal agent 1—Natamycin (Pimaricin) water (ABX+AFX-1): Baytril (enrofloxacin): 50 mg/kg/day; Metronidazole: 40 mg/kg/day; Vancomycin: 50 mg/kg/day; Natamycin (Pimaricin): 250 ug/kg/day

Group 6. Antibiotics plus Antifungal agent 2—Fluconazole water (ABX+AFX-2): Baytril (enrofloxacin): 50 mg/kg/day; Metronidazole: 40 mg/kg/day; Vancomycin: 50 mg/kg/day; Fluconazole: 100 mg/kg/day

Group 7. Antibiotics plus Antifungal agent 3—CRESEMBA water (AFX-3): (ABX+AFX-3): Baytril (enrofloxacin): 50 mg/kg/day; Metronidazole: 40 mg/kg/day; Vancomycin: 50 mg/kg/day; CRESEMBA (isavuconazonium sulfate): 200 mg/kg/day

Group 8. Control. Regular water with no antibiotics, no antifungal agents

Medicated water administration are continued for the weaned homozygous pups (Ednrb−/−) and wild type littermate controls (Ednrb+/+) until 23-25 days of life, at which point, the animals are euthanized via CO₂ followed by pneumothorax or by pneumothorax under anesthesia. The entire colon will be removed and a portion of the cecum and distal colon prepared for histopathological enterocolitis grading. The fresh feces will be collected from the cecum and distal colon after euthanasia and immediately freeze at −80° C. for batch processing of samples. The mouse fecal DNA and the DNA from the mouse chow in a similar manner are prepared for Next Generation Sequencing approach for Bacterial and Fungal Community Profiling. Significant changes in makeup between the samples will be verified by quantitative PCR.

### Example 4

Newborn Ednrb−/− mice with colonic aganglionosis and wild type littermates (n=5-7 per group) were administered either combination antibiotics (ABX, enrofloxacin, metronidazole, vancomycin), or antifungals (AFX1, natamycin or AFX2, fluconazole) alone or in combination via drinking water and then euthanized at 25 days. Colons were harvested and mid-colon (ganglionic region) was collected for histological enterocolitis scoring. Stool was also collected, DNA isolated and ITS and 16S libraries prepared for Next Generation Sequencing using Illumina MiSeq platform to assess bacterial and fungal community composition. Groups were compared using unpaired t-test. Without being bound to any particular theory, the inventors believe that in addition to bacteria, intestinal fungi contribute to Hirschsprung-associated enterocolitis (HAEC).

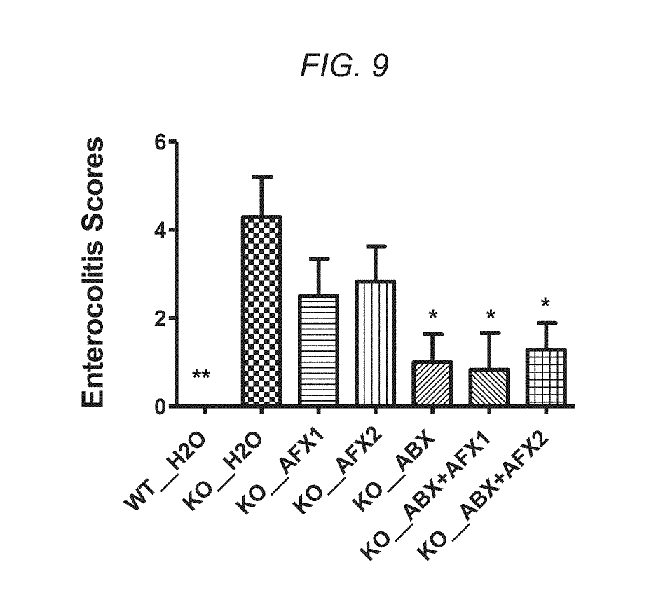

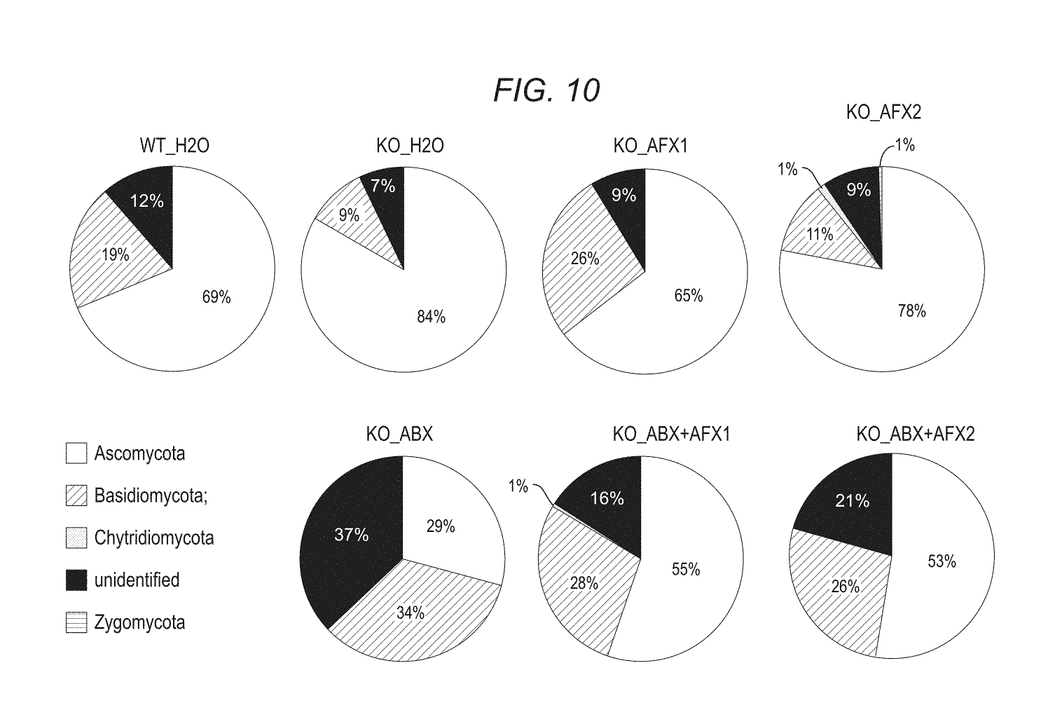

Enterocolitis scores were reduced in Ednrb−/− (KO) groups treated with antibiotics alone compared with untreated Ednrb−/− mice (FIG. 9). The antifungal only mice showed trends in reduced mean enterocolitis scores by 42% and 34%, respectively although they did not reach significance. The combination of antibiotics and antifungals, further decreased enterocolitis (FIG. 9). Interestingly, there was profound increase in proportion of “unidentified” fungi in the antibiotic treated group, which was partially ameliorated with the addition of either antifungal agent (FIG. 10).

Intestinal fungi contribute to enterocolitis in this murine model of HAEC and expansion of yet to be identified fungal species with antibiotic treatment. Without being bound to any particular theory, these findings open the possibility that Hirschsprung patients with refractory or recurrent HAEC after antibiotic treatment may benefit from the addition of antifungal therapy.

### Example 5

### Mouse Model of HAEC: Pull-Through Surgery in Ednrb-Null Mouse

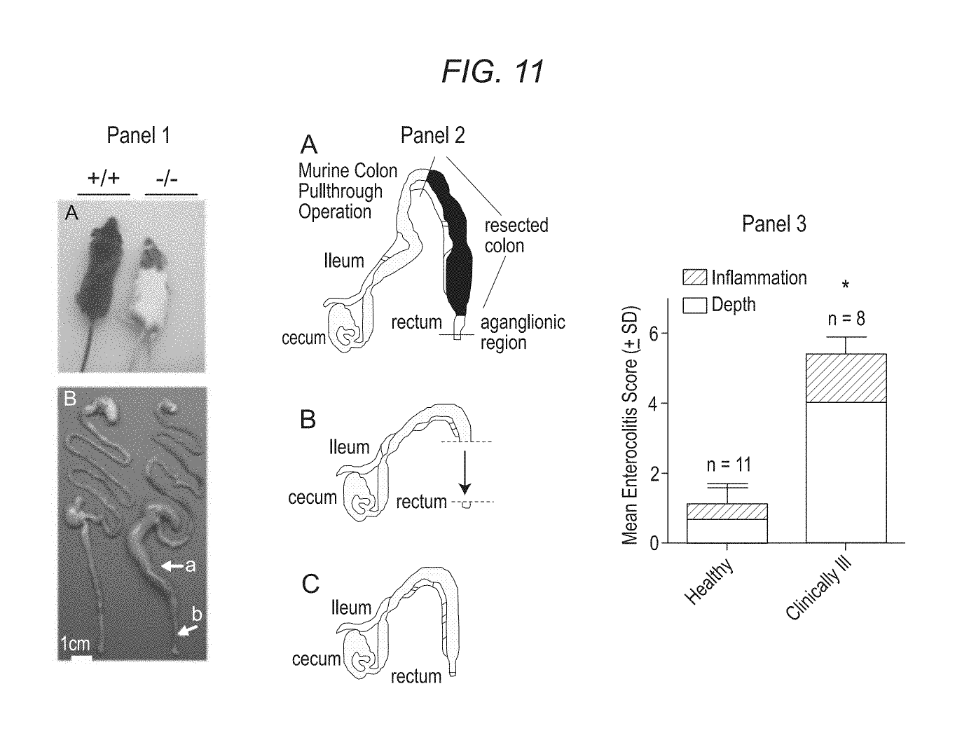

One of the challenges of studying disease mechanisms of HAEC was developing a suitable animal model that accurately mimics children with HAEC, most of whom develop it after pull-through surgery. Most Hirschsprung disease rodent models have a mutation in an HSCR-associated gene (e.g., Ednrb, Edn3, Sox10, Ret, etc.) that develop intestinal/colonic aganglionosis leaving the animals with an untreated intestinal obstruction from which the animals eventually succumb (FIG. 11 Panel 1). The Ednrb-null mouse is used most commonly, and is readily distinguishable from wild type and heterozygous littermates by its irregular white spotted coat due to the neural crest defect. The inventors developed a microsurgical pull-through operation which removed the obstructing aganglionic colonic segment in the Ednrb-null mouse, thereby rescuing it from lethality and creating a robust animal model closely analogous to humans treated for HSCR (FIG. 11 Panel 2). The inventors showed that approximately 40% of post-surgical Ednrb-null mice developed HAEC clinically and histologically, remarkably similar to the children who develop HAEC (FIG. 11 Panel 3). The inventors are currently using this model to investigate the gut fungal and bacterial community dynamics in the development of HAEC, as described herein.

Various embodiments of the invention are described above in the Detailed Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventors that the words and phrases in the specification and claims be given the ordinary and accustomed meanings to those of ordinary skill in the applicable art(s).

The foregoing description of various embodiments of the invention known to the applicant at this time of filing the application has been presented and is intended for the purposes of illustration and description. The present description is not intended to be exhaustive nor limit the invention to the precise form disclosed and many modifications and variations are possible in the light of the above teachings. The embodiments described serve to explain the principles of the invention and its practical application and to enable others skilled in the art to utilize the invention in various embodiments and with various modifications as are suited to the particular use contemplated. Therefore, it is intended that the invention not be limited to the particular embodiments disclosed for carrying out the invention.

While particular embodiments of the present invention have been shown and described, it will be obvious to those skilled in the art that, based upon the teachings herein, changes and modifications may be made without departing from this invention and its broader aspects and, therefore, the appended claims are to encompass within their scope all such changes and modifications as are within the true spirit and scope of this invention. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.).

What is claimed is:

1. A method of treating Hirschsprung-associated enterocolitis, comprising:
providing an antifungal agent; and
administering the antifungal agent to a subject in need thereof.

2. The method of claim 1, wherein the antifungal agent is isavuconazonium sulfate, posaconazole, itraconazole, efinaconazole, tavaborole, luliconazole, terbinafine, auriclosene, E-1224, VT-1161, NDV-3, NDV-3A, SQ-109, MGCD-290, ME-1111, LACTIN-V, or combinations thereof, or salts thereof.

3. The method of claim 1, wherein the antifungal agent is natamycin, fluconazole or a combination thereof, or salts thereof.

4. The method of claim 1, wherein the Hirschsprung-associated enterocolitis is caused, at least in part, by a fungus.

5. The method of claim 1, wherein the Hirschsprung-associated enterocolitis is caused, at least in part, by Candida.

6. The method of claim 1, further comprising providing an antibiotic agent and administering the antibiotic agent. The method of claim 6, wherein the antibiotic agent is selected from the group consisting of metronidazole, vancomycin, gentamicin, ciprofloxacin, levofloxacin and combinations thereof.

8. The method of claim 6, wherein the antibiotic agent is a combination of piperacillin and tazobactam.

9. A method of diagnosing Hirschsprung-associated enterocolitis, comprising:
obtaining a biological sample;
quantitating the amount of a fungal species in the biological sample;
diagnosing Hirschsprung-associated enterocolitis when the quantity of the fungal species is higher than a reference value.

10. A method of selecting a treatment for a subject suspected of having Hirschsprung-associated enterocolitis, comprising
obtaining a biological sample;
quantitating the amount of a fungal species in the biological sample;
selecting an antifungal treatment for the Hirschsprung-associated enterocolitis when the quantity of the fungal species is higher than a reference value.

11. The method of claim 10, quantitating the amount of a fungal species in the biological sample comprises using quantitative polymerase chain reaction (qPCR) detection of the amount of the fungal species.

12. The method of claim 10, wherein the fungal species is Candida species.

13. The method of claim 10, wherein the fungal species is Saccharomyces species.

14. The method of claim 12, wherein quantitating the amount of a fungal species in the biological sample comprises using quantitative polymerase chain reaction (qPCR) detection of the amount of the fungal species and TTTATCAACTTGTCACACCAGA (SEQ ID NO:1) (Forward) and ATCCCGCCTTACCACTACCG (SEQ ID NO:2) (Reverse) are used for C. albicans, and CAATCCTACCGCCAGAGGTTAT (SEQ ID NO:3) (Forward) and TGGCCACTAGCAAAATAAGCG (SEQ ID NO:4) (Reverse) are used for C. tropicalis.

15. The method of claim 12, wherein quantitating the amount of a fungal species in the biological sample comprises using quantitative polymerase chain reaction (qPCR) detection of the amount of the fungal species and one or more primers selected from the group consisting of SEQ ID NO:1, SEQ ID NO:2, SEQ ID NO:3, SEQ ID NO:4, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20.

16. The method of claim 10, wherein the antifungal treatment comprises an antifungal agent.

17. The method of claim 16, wherein the antifungal agent is selected from the group consisting of natamycin, fluconazole, isavuconazonium sulfate, posaconazole, itraconazole, efinaconazole, tavaborole, luliconazole, terbinafine, auriclosene, E-1224, VT-1161, NDV-3, NDV-3A, SQ-109, MGCD-290, ME-1111, LACTIN-V, combinations thereof, and salts thereof.

18. The method of claim 10, further comprising selecting an antibiotic.

19. The method of claim 18, wherein the antibiotic is selected from the group consisting of metronidazole, vancomycin, gentamicin, ciprofloxacin, levofloxacin and combinations thereof.

20. The method of claim 18, wherein the antibiotic is a combination of piperacillin and tazobactam.


SEQUENCE LISTING (20 sequences)

SEQ ID NO: 1 - DNA, 22 bp, Artificial Sequence
  Primer
tttatcaacttgtcacaccaga

SEQ ID NO: 2 - DNA, 20 bp, Artificial Sequence
  Primer
atcccgccttaccactaccg

SEQ ID NO: 3 - DNA, 22 bp, Artificial Sequence
  Primer
caatcctaccgccagaggttat

SEQ ID NO: 4 - DNA, 22 bp, Artificial Sequence
  Primer
tggccactagcaaaataagcgt

SEQ ID NO: 5 - DNA, 20 bp, Artificial Sequence
  Primer
agagtttgatcmtggctcag

SEQ ID NO: 6 - DNA, 15 bp, Artificial Sequence
  Primer
ctgctgcctyccgta

SEQ ID NO: 7 - DNA, 22 bp, Artificial Sequence
  Primer
cttggtcatttagaggaagtaa

SEQ ID NO: 8 - DNA, 20 bp, Artificial Sequence
  Primer
gctgcgttcttcatcgatgc

SEQ ID NO: 9 - DNA, 18 bp, Artificial Sequence
  Primer
cctgtttgagcgtcgttt

SEQ ID NO: 10 - DNA, 18 bp, Artificial Sequence
  Primer
tcctccgcttattgatat

SEQ ID NO: 11 - DNA, 17 bp, Artificial Sequence
  Primer
agattaaactcaaccaa

SEQ ID NO: 12 - DNA, 24 bp, Artificial Sequence
  Primer
cctatccattagtttatactccgc

SEQ ID NO: 13 - DNA, 17 bp, Artificial Sequence
  Primer
aggagtgcggttctttg

SEQ ID NO: 14 - DNA, 21 bp, Artificial Sequence
  Primer
tacttaccgaggcaagctaca

SEQ ID NO: 15 - DNA, 20 bp, Artificial Sequence
  Primer
attggagggcaagtctggtg

SEQ ID NO: 16 - DNA, 20 bp, Artificial Sequence
  Primer
ccgatccctagtcggcatag

SEQ ID NO: 17 - DNA, 22 bp, Artificial Sequence
  Primer
cttggtcatttagaggaagtaa

SEQ ID NO: 18 - DNA, 20 bp, Artificial Sequence
  Primer
gctgcgttcttcatcgatgc

SEQ ID NO: 19 - DNA, 21 bp, Artificial Sequence
  Primer
tttggtggcgggagcaatcct

SEQ ID NO: 20 - DNA, 24 bp, Artificial Sequence
  Primer
cgatgcgagaaccaagagatccgt